(19) United States
(12) Patent Application Publication
(10) Pub. No.: US 2021/0349028 A1
(43) Pub. Date: Nov. 11, 2021

(54) APPARATUS AND METHOD FOR DISPLAYING AND/OR PRINTING IMAGES OF A SPECIMEN INCLUDING A FLUOROPHORE

(71) Applicant: LEICA MICROSYSTEMS CMS GMBH, Wetzlar (DE)

(72) Inventor: Patric PELZER, Wetzlar (DE)

(21) Appl. No.: 17/308,172

(22) Filed: May 5, 2021

(30) Foreign Application Priority Data
May 8, 2020 (DE) 102020112572.0

(51) Int. Cl. G01N 21/64

(52) U.S. Cl. CPC G01N 21/6458; G01N 2021/6421; G01N 2021/6423; G01N 21/6428

(57) ABSTRACT

An apparatus for displaying and/or printing images of a specimen, including a first and second fluorophores and a second fluorophore, comprises an image acquisition unit configured to acquire first and second raw images captured by detecting first and second fluorescence light, having different spectral compositions, emitted by the first and second fluorophores, respectively. Each of the raw images comprises pixels each having a brightness value. The image processor is configured to: select first and second hues, that are different from each other, from a predetermined sequence of hues; generate a first false color image by assigning the first hue to each pixel of the first raw image; and generate a second false color image by assigning the second hue to each pixel of the second raw image. The output unit is configured to display and/or print the first and second false color images.

### CROSS-REFERENCE TO PRIOR APPLICATION

Priority is claimed to German Patent Application No. DE 10 2020 112 572.0, filed on May 8, 2020, the entire disclosure of which is hereby incorporated by reference herein.

### FIELD

The invention relates to an apparatus for displaying and/or printing images of a specimen including a fluorophore. The invention further relates to a method for displaying and/or printing images of a specimen including a fluorophore.

### BACKGROUND

Fluorescence microscopes comprise detection units configured to detect florescence light emitted by fluorophores introduced into a specimen in order to capture an image of the specimen. These detection units typically capture greyscale or monochromatic images. If more than one type of fluorophore is included within the specimen, optical filters are arranged in front of the detection unit in order to detect only the fluorescence light emitted by one of the fluorophores. Several images can be captures for different optical filter. Typically, one image for each type of fluorophore is acquired. However, since the detection unit only captures greyscale or monochromatic images. Even a trained observer will not always be able to infer which fluorophore was imaged based solely on the image content. As a means to ease the identification of the monochromatic images, these are often enhanced by false color-coding. False color-coding becomes even more important when the direct relation of different fluorophores needs to be compared by overlaying two or more images. In this case, only different false colors can be used as means for allowing to identify the fluorophore used.

### SUMMARY

In an embodiment, the present disclosure provides an apparatus for displaying and/or printing images of a specimen, including a first fluorophore and a second fluorophore. The apparatus comprises an image acquisition unit, an image processor and an output unit. The image acquisition unit is configured to acquire a first raw image captured by detecting first fluorescence light emitted by the first fluorophore and a second raw image captured by detecting second fluorescence light emitted by the second fluorophore. The second fluorescence light has a spectral composition that is different from a spectral composition of the first fluorescence light. Each of the first and second raw images comprises a plurality of pixels each having a brightness value. The image processor is configured to: select a first hue and a second hue from a predetermined sequence of hues, the first and second hues being different from each other; generate a first false color image by assigning the first hue to each of the pixels of the first raw image; and generate a second false color image by assigning the second hue to each of the pixels of the second raw image. The output unit is configured to display and/or print the first and second false color images.

### BRIEF DESCRIPTION OF THE DRAWINGS

Subject matter of the present disclosure will be described in even greater detail below based on the exemplary figures. The invention defined by the following claims is not limited to the exemplary embodiments. All features described and/or illustrated herein can be used alone or combined in different combinations. The features and advantages of various embodiments will become apparent by reading the following detailed description with reference to the attached drawings, which illustrate the following:

FIG. 1 is a schematic diagram of an apparatus for displaying images of a specimen and a fluorescence microscope;

FIG. 2 is a schematic diagram of raw images received by the apparatus, and false color images and the combined image generated by the apparatus according to FIG. 1; and

FIG. 3 a schematic diagram of the raw images received by the apparatus and first and second color-marked images generated by the apparatus according to FIG. 1.

### DETAILED DESCRIPTION

Embodiments of the present invention provide an apparatus and a method for displaying and/or printing images of a specimen including at least one fluorophore, that allows a user to distinguish and identify the features within the specimen, which are associated with the at least one fluorophore.

According to an embodiment, an apparatus for displaying and/or printing images of a specimen, including a first fluorophore and a second fluorophore, comprises an image acquisition unit. The image acquisition unit is configured to acquire a first raw image captured by detecting first fluorescence light emitted by the first fluorophore and a second raw image captured by detecting second fluorescence light emitted by the second fluorophore. The second fluorescence light has a spectral composition being different from a spectral composition of the first fluorescence light. Each of the first and second raw images comprises a plurality of pixel, each pixel having a brightness value. The apparatus also comprises an image processing unit (also referred to as an image processor) configured to select a first hue and a second hue from a predetermined sequence of hues, to generate a first false color image by assigning the first hue to each pixel of the first raw image, and to generate a second false color image by assigning the second hue to each pixel of the second raw image. The first and second hues are different from each other. The apparatus further comprises an output unit configured to display and/or print the first and second false color images.

Hue is a property of a color. In particular, a color can be uniquely described by its hue, its brightness and its saturation.

The apparatus acquires the first and second images by capturing the fluorescence light emitted by the fluorophores. These images may be greyscale or monochrome images, since the detectors used to acquire images of fluorophores typically do not measure the wavelength of the emitted fluorescence light. The raw images show different features of the specimen, each feature being associated with either the first fluorophore or the second fluorophore. The apparatus then colors the first and second images in the first and second hue, respectively. This may be done by changing the hue of each pixel of the first and second raw images to the first and second hue, respectively, while retaining the brightness value. Alternatively, the brightness value may be changed as well. For example, the brightness value of each pixel of the first and second raw images may be adjusted according to a predetermined functional relationship or a predetermined lookup table. Thereby, the first and second false color images are obtained. The first and second false color images are monochromatic images in the first and second hues, respectively, and can now be distinguished by their respective hue. This allows a user to distinguish and identify the features within the specimen, which are associated with the first and second fluorophores.

Alternatively or additionally, lookup tables may be used to adjust the first and second hues according to the brightness value. This can be used to enhance contrast between different brightness values, e.g. by using a maximally discontinuous lookup table such as a Glasbey lookup table.

In a preferred embodiment the sequence is predetermined to maximize a contrast between the hues of the sequence. For example, the hues of the sequence may be equidistantly arranged on the color-wheel. This makes it easier for the user to distinguish the features within the specimen associated with the first and second fluorophores.

In another preferred embodiment the first hue is determined based on the spectral composition of the first fluorescence light and the second hue is determined based on the spectral composition of the second fluorescence light. For example, if the first fluorophore emits predominately blue fluorescence light and the second fluorophore emits predominately red fluorescence light, the first hue is determined to be a blue hue and the second hue is determined to be a red hue. In particular, the first and second hues are chosen according to the perceived hue of the first and second fluorescence light, respectively. Alternatively, the first and second hues are chosen according to a wavelength of the emissions maximum of the first and second fluorophores, respectively. In this embodiment, the user can easily relate the features of the specimen in the first and second false color images to the different fluorophores.

In another preferred embodiment at least one of the first and second hues is determined based on at least one optical filter used for acquiring at least one of the first or second raw images. For example, the first or second hue are chosen to correspond to a wavelength of the transmission maximum of the optical filter used for acquiring the first or second raw image, respectively. Preferably, the apparatus is configured to acquire the information required to determine the first and/or second hues, e.g. the transmission maximum of the optical filter, from the optical filter. For example, the optical filter might be provided with an identifier, e.g. bar code or an RFID-chip, identifying the optical filter. In this embodiment, no action by the user, such as a user input, is required in order to relate the features of the specimen in the first and second false color images with the different fluorophores. This greatly enhances user friendliness of the apparatus.

In another preferred embodiment the sequence is predetermined such that hues of the sequence are suitable to be distinguished by a user with impaired color discrimination. In this embodiment the sequence is predetermined such, that is it possible for people with impaired color discrimination to unambiguously distinguish the colors of the sequence. This allows a user with impaired color discrimination to easily distinguish features within the specimen associated with the different fluorophores.

In another preferred embodiment the apparatus comprises a user input unit configured to receive a user input. The sequence is determined by the user input. In particular, the sequence is selected from a plurality of sequences by the user input. For example, the user may choose a sequence with maximized contrast between the hues in order to distinguish the features of the specimen associated with different fluorophores. In this embodiment, the user can select the sequence according to their individual needs allowing for greater flexibility.

Alternatively, or additionally, the user input comprises at least one identifier for identifying at least one of the first and second fluorophores, and/or for identifying the spectral composition of at least one of the first and second fluorescence light. Thus, allowing the user to relate the features of the specimen with the different fluorophores.

In another preferred embodiment the image processing unit is configured to combine the first and second false color images into a single combined image. The output unit is configured to display and/or print the combined image. In this embodiment an image of the specimen is displayed or printed as the single combined image. Thus, the user can see the features of the specimen associated with the different fluorophores in relation to each other.

Preferably, the output unit is configured to display and/or print the combined image and the first and second false color images simultaneously. This allows for distinguishing features of the specimen associated with the different fluorophores even if they overlap in the combined image.

In another preferred embodiment the output unit is configured to display and/or print the combined image and the first and second raw images simultaneously. In particular, if the first and second raw images are greyscale images, the contrast of the first and second raw images will be higher than the contrast of the combined image. Thus, in this embodiment, the user can distinguish even faint features of the specimen associated with the different fluorophores.

In another preferred embodiment the image processing unit is configured to generate a first color-marked image by adding a first color marker to the first raw image, in particular by arranging a first colored frame around the first raw image. The first color marker having the first hue. The image processing unit is further configured to generate a second color-marked image by adding a second color marker to the second raw image, in particular by arranging a second colored frame around the second raw image. The second color marker frame having the second hue. The output unit is configured to display and/or print the first and second color-marked images. The color markings make it easy to relate the first and second raw images to their associated fluorophore without coloring them. Thereby, the high contrast of the raw images is preserved while still allowing to distinguish the features of the specimen associated with the different fluorophores.

Optionally, the image acquisition unit is configured to receive the first and second raw images from a fluorescence microscope, in particular by means of a digital telecommunications network. This network might be the internet or a local area network. This embodiment allows the apparatus to be separate from fluorescence microscope enhancing the ease of use.

Another embodiment of the present invention provides an apparatus for displaying and/or printing images of a specimen including a fluorophore. The apparatus comprises: An image acquisition unit, configured to acquire a raw image captured by detecting fluorescence light emitted by the fluorophore, wherein the raw image comprises a plurality of pixel, each pixel having a brightness value. An image processing unit, configured to determine a hue based on the spectral composition of the fluorescence light, and to generate a false color image by assigning a single hue to each pixel of the raw image. An output unit, configured to display and/or print the false color image.

The hue is determined based on the spectral composition of the fluorescence light. This means, for example, if the fluorophore emits predominately blue fluorescence light, the hue is determined to be a blue hue. If on the other hand the fluorophore emits predominately red fluorescence light, the hue is determined to be a red hue. In particular, the hue is chosen according to the perceived hue of the fluorescence light. Alternatively, the hue is chosen according to a wavelength of the emissions maximum of the fluorophore. Thus, the user can easily relate the features of the specimen in the false color image to the specific type of fluorophore used.

In a preferred embodiment the image processing unit is configured to determine the hue based on an optical filter used for acquiring the raw image. Preferably, the apparatus is configured to acquire the information required to determine the hue, e.g. the transmission maximum of the optical filter, from the optical filter. For example, the optical filter might be provided with an identifier, e.g. bar code or an RFID-chip, identifying the optical filter. In this embodiment, no action by the user, such as a user input, is required in order to determine the optical filter used. This greatly enhances user friendliness of the apparatus.

An embodiment of the invention further relates to a method for displaying and/or printing an image of a specimen including a first fluorophore and a second fluorophore. The method comprises the following steps: Acquiring a first raw image captured by detecting first fluorescence light emitted by the first fluorophore. Acquiring a second raw image captured by detecting second fluorescence light emitted by the second fluorophore. The second fluorescence light has a spectral composition being different from a spectral composition of the first fluorescence light. The first and second raw images each comprise a plurality of pixel, each pixel having a brightness value. Selecting a first hue and a second hue from a predetermined sequence of hues. Generating a first false color image by assigning the first hue to each pixel of the first raw image. Generating a second false color image by assigning the second hue to each pixel of the second raw image, wherein the first and second hues are different from each other. Displaying and/or printing the first and second false color images.

Embodiments of the method have the same advantages as embodiments of the apparatus and can be supplemented using the features of any of the embodiments of the apparatus.

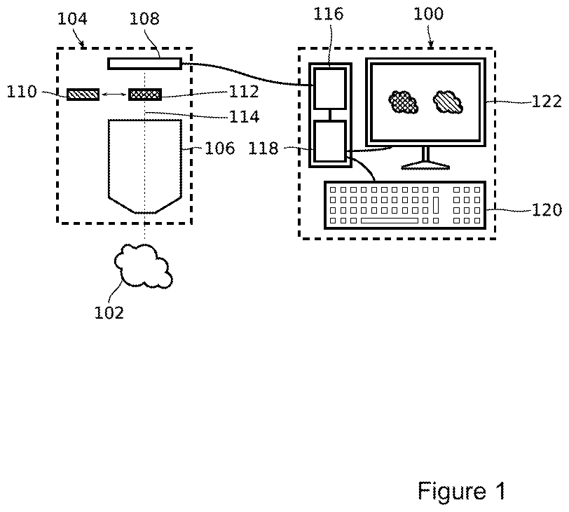

FIG. 1 shows a schematic diagram of an apparatus 100 for displaying images of a specimen 102 including a first fluorophore and a second fluorophore according to an embodiment. FIG. 1 further shows a fluorescence microscope 104 configured to capture images of the specimen 102.

The fluorescence microscope 104 comprises an optical system 106 configured to image the specimen 102 onto a detection unit 108. The fluorescence microscope 104 further comprises a set of exchangeable optical filters 110, 112 that can be brought into a light path 114 of the fluorescence microscope 104. A first optical filter 110 of the exchangeable optical filters 110, 112 is only transparent to first fluorescence light emitted by the first fluorophore. A second optical filter 112 of the exchangeable optical filters 110, 112 is only transparent to second fluorescence light emitted by the second fluorophore. Alternatively, the fluorescence microscope 104 may comprise other means for selecting specific wavelengths, e.g. a dispersion element such as a grating.

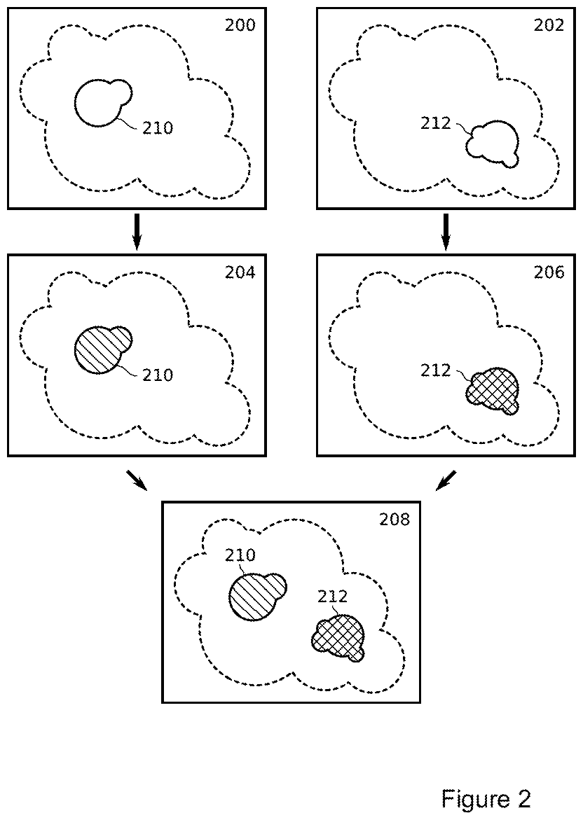

By introducing the first optical filter 110 into the light path 114 of the fluorescence microscope 104, only features of the specimen 102 including the first fluorophore are imaged by the detection unit 108. Accordingly, by introducing the second optical filter 112 into the light path 114 of the fluorescence microscope 104, only features of the specimen 102 including the second fluorophore are imaged. The features of the specimen 102 comprising the first fluorophore are henceforth called first features 210 (see FIG. 2) and the features of the specimen 102 comprising the second fluorophore are henceforth called second features 212 (see FIG. 2). The images of the first and second features 210, 212 captured by the detection unit 108 are called first and second raw images 200, 202 (see FIG. 2), respectively.

Alternatively, the first and second features 210, 212 are imaged by first exciting only the first fluorophore, e.g. with a laser having a first wavelength, and capturing the first raw image 200, and then exciting only the second fluorophore, e.g. with a laser having a second wavelength, and capturing the second raw image 202. In this embodiment, the set of exchangeable filters 110, 112 is not needed.

The apparatus 100 comprises an image acquisition unit 116, an image processing unit 118, a user input unit 120, and an output unit 122. The image acquisition unit 116 is configured to receive the first and second raw images 200, 202 from the fluorescence microscope 104. The image processing unit 118 is configured to generate first and second false color images 204, 206 (see FIG. 2) from the first and second raw images 200, 202, respectively. In order to generate the first and second false color images 204, 206 the image processing unit 118 first selects a first hue and a second hue from a predetermined sequence of hues, the first and second hues being different from each other.

In the present embodiment, the first and second hues are determined based on the first and second optical filters 110, 112, respectively. This means, if e.g. the first optical filter 110 is transmissive to predominately blue light, the first hue is selected to be a blue hue. The first and second hues may also be selected based on the first and second fluorophores, respectively. For example, the specific type of the first fluorophore and the second fluorophore may be entered into the apparatus 100 by a user via the user input unit 120. The first and second hues are then selected based on the user input. In this embodiment the first and second hues are determined based on the spectral composition of the first and second fluorescence light, respectively.

In alternative embodiments, the first and second hues are determined independent from the spectral composition of the first and second fluorescence light. In an alternative embodiment, the first and second hues are selected from a sequence that is predetermined to maximize a contrast between the hues of the sequence. In another alternative embodiment, the first and second hues are selected from a sequence that is predetermined such that hues of the sequence are suitable to be distinguished by a user with impaired color discrimination. The selection of both the sequence and the first and second hues may be determined by a user input received via the user input unit 120.

The image processing unit 118 is configured to generate the first false color image 204 by assigning the first hue to each pixel of the first raw image 200, and to generate the second false color image 206 by assigning the second hue to each pixel of the second raw image 202. In the present embodiment, the brightness value of each pixel is kept. That is, the brightness value of each pixel in the first and second false color images 204, 206 is the same as the brightness level of the corresponding pixel in the first and second raw images 200, 202, respectively. Alternatively, the brightness level of each pixel may be adjusted according to a functional dependency or a lookup table. The image processing unit 118 also is configured to combine the first and second false color images 204, 206 into a single combined image 208 (see FIG. 2). The generation of the first and second false color images 204, 206 and the combined image 208 is shown in FIG. 2.

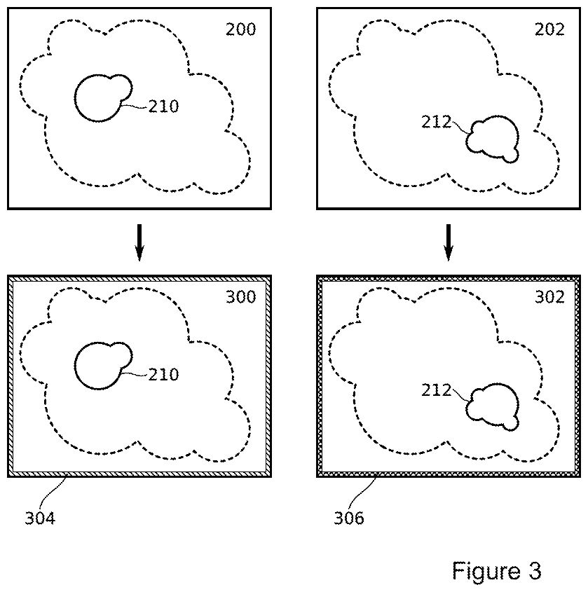

The image processing is further configured to generate to generate a first and second color-marked images 300, 302 (see FIG. 3). The first color-marked image 300 is generated by adding a first color marker 304 (see FIG. 3) having the first hue to the first raw image 200. The second color-marked image 302 is generated by adding a second color marker 306 (see FIG. 3) having the second hue to the second raw image 202. In the present embodiment, the first and second color markers 304, 306 are colored frames. The generation of the first and second color-marked images 300, 302 is shown in FIG. 3.

The output unit 122 is exemplary designed as a computer monitor and configured to display at least one of the first and second raw images 200, 202, the first and second false color images 204, 206, the first and second color-marked images 300, 302 and the combined image 208 depending on the user input received via the user input unit 120. The output unit 122 is further configured to display any combination of the aforementioned images depending on the user input received via the user input unit 120.

FIG. 2 shows a schematic diagram of the first and second raw images 200, 202 received by the apparatus 100. FIG. 2 furthers shows a schematic diagram of the first and second false color images 204, 206 and the combined image 208 generated by the apparatus 100. An outline of the specimen 102 is shown as a dashed line in FIG. 2.

The first raw image 200 comprises the first features 210, that is a feature of the specimen 102, e.g. a cell of cluster of cells, including the first fluorophore. The second raw image 202 comprises the second features 212, that is a feature of the specimen 102 different from the first features 210 including the second fluorophore. The first false color image 204 comprises the first features 210 colored in the first hue. The first hue is shown in FIG. 2 a hatch pattern. The second false color image 206 comprises the second features 212 colored in the second hue. The second hue is shown in FIG. 2 a crosshatch pattern. The combined image 208 comprises both the first and second features 210, 212.

FIG. 3 shows a schematic diagram of the first and second raw images 200, 202 received by the apparatus 100 and the first and second color-marked images 300, 302 generated by the apparatus 100.

The first color-marked image 300 comprises the first features 210 and the first color marker 304. The first color marker 304 is exemplary formed as a colored frame having the first hue. The second color-marked image 302 comprises the second features 212 and the second color marker 306. The second color marker 306 is exemplary formed as a colored frame having the second hue. Alternatively, the first and second color markers 304, 306 may take any other suitable form, e.g. a colored circle having the first or second hue, respectively, arranged in a corner of the respective color-marked image.

As used herein the term “and/or” includes any and all combinations of one or more of the associated listed items and may be abbreviated as “/”.

Although some aspects have been described in the context of an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus.

While embodiments of the invention have been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LIST OF REFERENCE SIGNS

100 Apparatus

102 Specimen

104 Fluorescence microscope

106 Optical system

108 Detection unit

110, 112 Optical filter

114 Light path

116 Image acquisition unit

118 Image processing unit

120 Unser input unit

122 Output unit

200, 202 Raw image

204, 206 False color image

208 Combined image

210, 212 Feature

300, 302 Color-marked image

304, 306 Color marker

What is claimed is:

1. An apparatus for displaying and/or printing images of a specimen including a first fluorophore and a second fluorophore, the apparatus comprising:
an image acquisition unit configured to acquire a first raw image captured by detecting first fluorescence light emitted by the first fluorophore and a second raw image captured by detecting second fluorescence light emitted by the second fluorophore, the second fluorescence light having a spectral composition that is different from a spectral composition of the first fluorescence light, wherein each of the first and second raw images comprises a plurality of pixels each having a brightness value;
an image processor configured to:
select a first hue and a second hue from a predetermined sequence of hues, the first and second hues being different from each other,
generate a first false color image by assigning the first hue to each of the pixels of the first raw image, and
generate a second false color image by assigning the second hue to each of the pixels of the second raw image; and
an output unit configured to display and/or print the first and second false color images.

2. The apparatus according to claim 1, wherein the sequence of hues is predetermined to maximize a contrast between the hues of the sequence.

3. The apparatus according to claim 1, wherein the first hue is determined based on the spectral composition of the first fluorescence light and the second hue is determined based on the spectral composition of the second fluorescence light.

4. The apparatus according to claim 1, wherein at least one of the first and second hues is determined based on at least one optical filter used for acquiring at least one of the first or second raw images.

5. The apparatus according to claim 1, wherein the sequence of hues is predetermined such that hues of the sequence are suitable to be distinguished by a user with impaired color discrimination.

6. The apparatus according to claim 1, further comprising a user input unit configured to receive a user input, wherein the sequence of hues is determined by the user input.

7. The apparatus according to claim 6, wherein the sequence of hues is selected from a plurality of sequences by the user input.

8. The apparatus according to claim 6, wherein the user input comprises at least one identifier for identifying at least one of the first and second fluorophores, and/or for identifying the spectral composition of at least one of the first and second fluorescence light.

9. The apparatus according to claim 1, wherein the image processor is configured to combine the first and second false color images into a single combined image, and wherein the output unit is configured to display and/or print the combined image.

10. The apparatus according to claim 9, wherein the output unit is configured to display and/or print the combined image and the first and second false color images simultaneously.

11. The apparatus according to claim 9, wherein the output unit is configured to display and/or print the combined image and the first and second raw images simultaneously.

12. The apparatus according to claim 1, wherein the image processor is configured to:
generate a first color-marked image by adding a first color marker to the first raw image, the first color marker having the first hue; and
generate a second color-marked image by adding a second color marker to the second raw image, the second color marker frame having the second hue, and
wherein the output unit is configured to display and/or print the first and second color-marked images.

13. The apparatus according to claim 12, wherein the first color-marked image is generated by arranging a first colored frame around the first raw image, and wherein the second color-marked image is generated by arranging a second colored frame around the second raw image.

14. The apparatus according to claim 1, wherein the image acquisition unit is configured to receive the first and second raw images from a fluorescence microscope.

15. The apparatus according to claim 14, wherein image acquisition unit is configured to receive the first and second raw images from the fluorescence microscope via a digital telecommunications network.

16. A method for displaying and/or printing an image of a specimen including a first fluorophore and a second fluorophore, the method comprising:
acquiring a first raw image captured by detecting first fluorescence light emitted by the first fluorophore and a second raw image captured by detecting second fluorescence light emitted by the second fluorophore, the second fluorescence light having a spectral composition that is different from a spectral composition of the first fluorescence light, wherein the first and second raw images each comprise a plurality of pixels each having a brightness value;
selecting a first hue and a second hue from a predetermined sequence of hues, the first and second hues being different from each other;
generating a first false color image by assigning the first hue to each of the pixels of the first raw image;
generating a second false color image by assigning the second hue to each of the pixels of the second raw image; and
displaying and/or printing the first and second false color images.

17. An apparatus for displaying and/or printing images of a specimen including a fluorophore, the apparatus comprising:
an image acquisition unit configured to acquire a raw image captured by detecting fluorescence light emitted by the fluorophore, wherein the raw image comprises a plurality of pixels each having a brightness value;
an image processor, configured to:
determine a hue based on a spectral composition of the fluorescence light, and
generate a false color image by assigning a single hue to each of the pixels of the raw image; and
an output unit configured to display and/or print the false color image.

18. The apparatus according to claim 17, wherein the image processor is configured to determine the hue based on an optical filter used for acquiring the raw image.

19. A method for displaying and/or printing images of a specimen including a fluorophore, the method comprising:
acquiring a raw image captured by detecting fluorescence light emitted by the fluorophore, wherein the raw image comprises a plurality of pixels each having a brightness value;
determining a hue based on a spectral composition of the fluorescence light;
generating a false color image by assigning a single hue to each of the pixels of the raw image; and
displaying and/or printing the false color image.
